(19) United States
(12) Patent Application Publication
(10) Pub. No.: US 2017/0197054 A1
(43) Pub. Date: Jul. 13, 2017

(54) FIXING TOOL FOR ATTACHING ARTIFICIAL VENTILATOR

(71) Applicant: IKK CO., LTD., Kahoku-shi, Ishikawa (JP)

(72) Inventors: Setsuo TAKIZAWA, Nomi-shi, Ishikawa (JP); Tomomi MAEDA, Komatsu-shi, Ishikawa (JP); Kazuhide ITO, Kahoku-shi, Ishikawa (JP)

(21) Appl. No.: 15/327,581

(22) PCT Filed: Jul. 3, 2015
(86) PCT No.: PCT/JP2015/069278

§ 371 Date: Jan. 19, 2017

(30) Foreign Application Priority Data
Oct. 16, 2014 (JP) 2014-211275
Mar. 17, 2015 (JP) 2015-052911

(51) Int. Cl. A61M 16/06

(52) U.S. Cl. CPC A61M 16/0683; A61M 16/0633

(57) ABSTRACT

A fixing tool for attaching an artificial ventilator includes a forehead-side fixing band 2 which is composed of a belt-like member made from a stretchable mesh cloth having multiple pores 1a formed therein and/or a belt-like member made from a stretchable cloth having a stretch rate of 140 to 180% and which can fix an artificial ventilator in a circumferential direction from the forehead through the head and a mouth-side fixing band 3 which can fix the artificial ventilator in a circumferential direction from the mouth through the neck. The forehead-side fixing band 2 and the mouth-side fixing band 3 are connected to each other by connection bands 4, 5 and 6 each being made from the same material as that of the mesh cloth.

### TECHNICAL FIELD

The present invention relates to a fixing tool for attaching an artificial ventilator for attaching/detaching the artificial ventilator to/from a front side of a face by using a fixing band (headgear).

### BACKGROUND ART

The artificial ventilator is a medical device for mechanically carrying out artificial respiration. When the artificial ventilator is used, it is realized by inserting/intubating a tracheal tube into an airway or by using a mask. A ventilation path used for the artificial ventilator includes the following three in general. They are tracheal intubation (the most rapid/reliable way for maintenance of airway in emergency or during an operation), tracheotomy (used for transtracheal artificial respiration control for a long time mainly of two weeks or more), and a mask (used for non-invasive positive pressure ventilation: NPPV). The non-invasive positive pressure ventilation here refers to a positive-pressure artificial respiration carried out without indwelling an artificial airway (tracheal tube) in a trachea. The mask for non-invasive positive pressure ventilation requires easy attachment/detachment and capability of self-control by a patient as in the case of a sleep apnea syndrome and the like.

Types of the mask for non-invasive positive pressure ventilation include, as illustrated in FIGS. 7A, 7B, and 7C, a nasal mask S1, a full-face mask S2, a total-face mask S3 and the like. Fixing tools (headgears) for attaching these artificial ventilators include a forehead-side fixing band (upper-side band) for fixation in a circumferential direction from a forehead to a head and a mouth-side fixing band (lower-side band) for fixation in the circumferential direction from a mouth to a neck part in many cases. A single fixing band is used in some cases, but in order to ensure reliability of connection with a pressure/oxygen port and connection with the artificial ventilator, a double band type which is more stable and hardly causes a displacement at an accurate position is on a mainstream.

Patent Literature 1 discloses the following:

a headgear assembly for maintaining a respiratory mask assembly in position on a patient's face, the headgear assembly comprising:

two side portions; and

a rear portion positioned between the side portions and the rear portion being structured and arranged to engage a lower portion of the occipital bone of the patient's head to prevent upward movement of the headgear assembly in use,

wherein each side portion comprises an upper side strap constructed and arranged to be located above the ear and eye of the patient in use, and a lower side strap constructed and arranged to be located below the ear and eye of the patient in use,

each said upper side strap and each said lower side strap are further constructed and arranged to be connectable to the respiratory mask assembly;

wherein the headgear assembly is constructed from a flexible material; and

the rear portion of the headgear comprises a stiffening portion configured to maintain the position of each upper side strap and each lower side strap spaced from the patient's ears in use (claim 1 thereof).

Moreover, Patent Literature 2 discloses the following:

A respiratory mask assembly for delivering breathable gas to a patient, comprising:

a frame having a main body and a side frame member provided on each lateral side of the main body, each side frame member including an integrally formed first connector portion;

a cushion removably attachable to the main body of the frame; and

a headgear assembly comprising a strap, the headgear assembly being removably attachable to the frame, the headgear assembly having a second connector portion provided on the strap, the second connector portion being adapted to be removably coupled with the first connector portion provided on the frame, the second connector portion comprising at least one fixing recess portion provided for engaging with a tooth portion provided on the frame so as to fix rotation of the headgear assembly at a plurality of predetermined rotation positions with respect to the cushion,

wherein the cushion includes a side wall having a first interlocking surface that engages a second interlocking surface provided by the frame when the cushion and frame are engaged with one another, wherein the first and second interlocking surfaces engaging with one another to removably attach the cushion to the frame, and

the headgear assembly is rotationally adjustable with respect to the frame (claim 1 thereof).

In Patent Literatures 1 and 2, their lengths are adjusted by using the strap by using a locking member (locking holder) Sa as illustrated in FIGS. 8A and 8B, but there is a problem that the attachment is not easy for a physically handicapped patient. Thus, instead of the locking member, those with a hook-and-loop fastener connected to a distal end side of the strap are conceived of. However, in the case of wearing for a long time, a pressure of the mask causes bedsores or problems of adhesion of the hook-and-loop fastener to anywhere or of prevention of stretchability occur as will be described later.

Patent Literature 3 discloses a three-dimensional assembly type mask displacement preventing tool, wherein an excess rubber strap of a mask is hooked and pinched on a rubber-strap retaining clip of the three-dimensional assembly type mask displacement preventing tool separated from the mask so that the mask is not displaced by utilizing friction resistance.

### PRIOR ART DOCUMENT

### Patent Literature

### [Patent Literature 1] Japanese Patent Laid-Open No. 2014-128713

### [Patent Literature 2] Japanese Patent No. 4544830

### [Patent Literature 3] Japanese Patent Laid-Open No. 2012-250003

### SUMMARY OF INVENTION

### Technical Problem

When the artificial ventilator is to be attached to a patient, the patient's posture is not the same, that is, the artificial ventilator is attached to the patient in a normal face-up posture on the bed or in a state close to lying on the side or face-down. The patient may be an adult or a child, and a person who wears is not the same, either. Even in such various states, the artificial ventilator should be attached accurately and rapidly without causing a displacement or the like.

However, in Patent Literature 3, since the excess rubber strap of the existing artificial ventilation mask is hooked and pinched on the rubber-strap retaining clip of the solid assembly mask holder, a general displacement can be caused easily by movement of the face of the patient. Moreover, Patent Literatures 1 and 2 include a forehead-side fixing band for fixation in the circumferential direction from the forehead to the head and the mouth-side fixing band for fixation in the circumferential direction from the mouth to the neck part, but with only them, there is a concern that the artificial ventilator is displaced in a peripheral direction and is moved. Moreover, the artificial ventilator needs to be attached accurately and rapidly to a position from the mouth to the nose on the front side of the face of the patient.

Moreover, the conventional fixing tool (headgear) for attaching an artificial ventilator is constituted by a stretchable member, but since attachment to an adult is assumed, when it is to be used for a child, due to an extreme difference in a size, it has a problem that the stretchability does not function effectively any more. In this regard, Patent Literature 1 is constituted by a soft and flexible material, but actually, a plurality of cloths are overlapped, and only stretchability of a leather product can be exerted, and it has a problem that air permeability is not sufficient. Moreover, although Patent Literature 1 is constituted by a soft and flexible material, since the hook-and-loop fastener adheres anywhere in the material, it adheres everywhere, and adhesion occurs even in an unexpected spot during attachment/detachment of the artificial ventilator, which results in a problem of interference with an operation of attachment/detachment. Furthermore, the fixing tool (headgear) for attaching an artificial ventilator is repeatedly used by washing, but an effect of washing is not much excellent, and it has a problem of bulkiness in the case of storing/keeping after the washing.

Thus, an object of the present invention is to provide a fixing tool for attaching an artificial ventilator which has an extremely high stretch rate and excellent air permeability, can be attached/detached easily, and can reliably attach/detach the artificial ventilator to/from a front side of a face and moreover, has an excellent washing effect and can be neatly folded and stored/kept.

### Solution to Problem

The present invention provides a fixing tool for attaching an artificial ventilator for attaching/detaching the artificial ventilator to/from a front side of a face by using a fixing band, the fixing band comprising: a forehead-side fixing band for fixation in a circumferential direction from a forehead to a head; a mouth-side fixing band for fixation in the circumferential direction from a mouth to a neck part; and a slip resistant member fixed to center positions of the forehead-side fixing band and the mouth-side fixing band and on which air-permeable pores are formed.

Moreover, in the fixing tool for attaching an artificial ventilator of the present invention, the slip resistant member is provided on the forehead-side fixing band and on the mouth-side fixing band so that the artificial ventilator is not displaced from the face front, and the air-permeable pores are formed on the slip resistant member.

According to the present invention, since the slip resistant member for preventing displacement of the artificial ventilator from the face front is provided, the artificial ventilator is reliably fixed on the front side of the face. In addition, air permeability is ensured since a large number of pores are formed on the slip resistant member, and stretchability (stretchability in vertical and horizontal directions) is improved by the large number of pores so that a wearing feeling is favorable and attachment without displacement of the artificial ventilator is realized even if a user moves.

Here, the slip resistant member is preferably provided at the front center positions of the forehead-side fixing band and the mouth-side fixing band so that the slip resistant member is not displaced from the head (occipital part).

According to the present invention, since the slip resistant member is fixed at the front center positions of the forehead-side fixing band and the mouth-side fixing band, the artificial ventilator is not easily displaced even if the user moves, which enables favorable attachment for a long time. Moreover, according to the present invention, since the slip resistant member is provided without contact with skin of the user, it is possible to use with a favorable wearing feeling without damaging the skin.

The present invention provides a fixing tool for attaching an artificial ventilator for attaching/detaching the artificial ventilator to/from the front side of the face by using a fixing band, the fixing band comprising: a forehead-side fixing band for fixation in the circumferential direction from a forehead to a head; and the mouth-side fixing band for fixation in the circumferential direction from the mouth to the neck part, and the fixing band being constituted by a belt-like member made of a stretchable mesh cloth on which a large number of pores are formed and/or being constituted by a belt-like member made of a stretchable cloth having a stretch rate of 100 to 300%.

Moreover, in the present invention, the fixing band is constituted by a belt-like member made of a stretchable mesh cloth on which a large number of pores are formed and/or being constituted by a belt-like member made of a stretchable cloth having a stretch rate of 100 to 300%.

Here, the stretch rate means an elongation rate at a 0.5 kg load (constant load method) described in JIS L1096. Here, a state in which no load is applied is assumed to be 100%.

According to the present invention, since the fixing band is constituted by a belt-like member made of a stretchable mesh cloth on which a large number of pores are formed and/or is constituted by a belt-like member made of a stretchable cloth having a stretch rate of 100 to 300%, the artificial ventilator can be attached/detached with extremely high stretchability. That is, air permeability is ensured by the mesh cloth on which a large number of pores are formed, and on the other hand, the large number of pores also influence stretchability. Similarly, the stretchability of the stretchable cloth having the stretch rate of 100 to 300% becomes extremely high and is excellent for attachment of the artificial ventilator.

The fixing band of the present invention further comprises at least one of connection bands, that is: a head rear-side connection band fixed substantially perpendicularly at the vicinity of the centers of the forehead-side fixing band and the mouth-side fixing band and disposed so as to span from the forehead-side fixing band to the mouth-side fixing band; a head side connection band for connecting the forehead-side fixing band so as to cover the head; a side connection band for connecting the forehead-side fixing band and the mouth-side fixing band; or a jaw-side connection band for connecting the mouth-side fixing band so as to cover a jaw part, each of these connection bands being constituted by a belt-like member made of a stretchable mesh cloth on which a large number of pores are formed and/or being constituted by a belt-like member made of a stretchable cloth having the stretch rate of 100 to 300%.

According to the present invention, each of these connection bands is constituted by the belt-like member made of the stretchable mesh cloth on which a large number of pores are formed and/or is constituted by the belt-like member made of a stretchable cloth having the stretch rate of 100 to 300%. That is, by combination with each of the connection bands, the fixing tool according to the present invention can be assembled so that stretchability is exerted not only in vertical and horizontal directions but also in a diagonal direction, and that the artificial ventilator is disposed on the front of the face by wearing it at a time like a cap and is also removed at a time.

In the present invention, the connection band is attached/detached via connecting means such as a hook-and-loop fastener.

According to the present invention, since a spot for attachment/detachment using the hook-and-loop fastener is predefined and attachment/detachment does not occur at a spot other than that, adhesion at another unexpected spot does not occur during an attachment operation as conventionally, and thus the operation is facilitated. The mesh cloth can be made to act such that the hook-and-loop fastener does not adhere easily to a spot having a large number of pores.

Moreover, since the forehead-side fixing band and the mouth-side fixing band are constituted separably, and connected via the connecting means such as the hook-and-loop fastener, length can be easily adjusted and accurate attachment to the front of the face without a displacement is realized even if the size must be significantly varied for, for example, use by an adult and use by a child.

Moreover, in the present invention, the forehead-side fixing band and the mouth-side fixing band are attached/detached by the connection band for connecting them only on one sides thereof via the connecting tool such as the hook-and-loop fastener, and the other sides are connected in advance by sewing or the like.

According to the present invention, since connection is made only on one side by the connection band, an operation is easier than the case where both sides of the connection band.

In the present invention, right and left side connection bands are provided for connecting the forehead-side fixing band and the mouth-side fixing band on the right and left sides of the face, and a rear-side connection band for connecting the head rear-side connection band is provided for connecting the forehead-side fixing band on the head side and a rear-side connection band for connecting the forehead-side fixing band and the mouth-side fixing band on the rear side of the head.

According to the present invention, since the head side connection band is provided for connecting the forehead-side fixing band disposed in the circumferential direction on the head side, there is no more concern that the artificial ventilator is displaced to the head side. Moreover, by connecting the head side connection band for connecting the forehead-side fixing band disposed in advance in the circumferential direction on the head side, attachment/detachment like a cap is worn is realized. Moreover, since the right and left side connection bands are provided for connecting the forehead-side fixing band and the mouth-side fixing band on the right and left sides of the face, a displacement of the artificial ventilator on the front side of the face can be made difficult to occur.

Moreover, the fixing tool for attaching an artificial ventilator of the present invention is used for treatment of a patient with an apnea syndrome.

According to the experiments in Example 2, Example 3, and Example 4 which will be described later, applying the fixing tool for attaching an artificial ventilator of the present invention to an apnea syndrome patient resulted in appropriate fit of the mask onto the face due to its extremely excellent stretchability than the conventional fixing tools for attaching an artificial ventilator so that the mask could be hardly displaced and air leakage (leak amount) from the mask was decreased, and thus the apnea hypopnea index or apnea index (AHI, AI) was decreased. The fixing tool for attaching an artificial ventilator of the present invention is optimal for treatment of an apnea syndrome patient.

### Advantageous Effects of Invention

According to the fixing tool for attaching an artificial ventilator of the present invention, air permeability is ensured by the mesh cloth, and the washing effect is also excellent. Moreover, since the cloth with high stretchability used is the mesh cloth, more stretching force is produced so that the artificial ventilator can be attached/detached with remarkable stretchability. Moreover, according to the present invention, since the spot for attachment/detachment using the connecting means such as the hook-and-loop fastener is predefined and attachment/detachment does not occur at a spot other than that, adhesion at another unexpected spot does not occur during the attachment operation as conventionally, and thus the operation is facilitated. Moreover, since the forehead-side fixing band and the mouth-side fixing band in the separation constitution are connected by using the connection band, it can be formed into a shape to be worn on the head, and by bringing the right and left connection bands on the face to the front side other than the upper and lower sides of the face, and moreover, according to the present invention, since a forehead fixing portion and a mouth fixing portion are constituted separably, by connecting them with adjusting the length by the connecting means such as the hook-and-loop fastener, attachment fitting the size of the face of the patient can be realized. That is, whether the patient is an adult or a child, the length can be adjusted by connection from the separated state. Moreover, by attaching the forehead-side fixing band and the mouth-side fixing band to the artificial ventilator and then connecting a head band and the right and left-side bands with adjusting the length via the connecting means such as the hook-and-loop fastener, attachment fitting the size of the face is realized by an operation equivalent to the conventional operation without causing a displacement on the front side of the face of the patient.

Moreover, according to the fixing tool for attaching an artificial ventilator of the present invention, since the slip resistant member for preventing displacement of the artificial ventilator from the face front is provided, the artificial ventilator is reliably fixed on the front side of the face. In addition, air permeability is ensured since a large number of pores are formed on the slip resistant member, and stretchability (stretchability in vertical and horizontal directions) is improved by the large number of pores so that a wearing feeling is favorable and attachment without displacement of the artificial ventilator is realized even if a user moves.

Moreover, since the slip resistant member is provided at the front center positions of the forehead-side fixing band and the mouth-side fixing band so as not to slip from the head (occipital part), the artificial ventilator is not displaced easily even if the user moves, which enables favorable attachment for a long time. Moreover, since the slip resistant member is provided without contact with skin of the user, it is possible to use with a favorable wearing feeling without damaging the skin.

### BRIEF DESCRIPTION OF THE DRAWINGS

FIG. 1 are views illustrating a fixing tool for attaching an artificial ventilator of a first embodiment to which the present invention is applied, in which FIG. 1A is a front view thereof, FIG. 1B is a side view thereof, and FIG. 1C is a rear view thereof;

FIG. 2 is a development view of the fixing tool for attaching an artificial ventilator of the first embodiment;

FIG. 3 is a plan view of the fixing tool for attaching an artificial ventilator of the first embodiment;

FIG. 4 is a plan view of the fixing tool for attaching an artificial ventilator of the first embodiment;

FIG. 5 is a plan view of the fixing tool for attaching an artificial ventilator of the first embodiment;

FIG. 6 are views illustrating a fixing tool for attaching an artificial ventilator of a second embodiment to which the present invention is applied, in which FIG. 6A is a plan view thereof and FIG. 6B is a side view thereof in a use state;

FIG. 7 is a view for explaining an example of a medical artificial ventilator;

FIG. 8 is a plan view illustrating a fixing tool for attaching an artificial ventilator of the second embodiment to which the present invention is applied;

FIG. 9 is a plan view illustrating a shape of each of a large number of pores in the first and second embodiments;

FIG. 10 is a rear view illustrating a fixing tool for attaching an artificial ventilator of a third embodiment to which the present invention is applied;

FIG. 11 is a front view illustrating a fixing tool for attaching an artificial ventilator of the third embodiment;

FIG. 12 is a perspective view of a state where the fixing tool for attaching an artificial ventilator of the third embodiment is attached;

FIG. 13 is a side view of a state where the fixing tool for attaching an artificial ventilator of the third embodiment is attached;

FIG. 14 is a rear view of a state where the fixing tool for attaching an artificial ventilator of the third embodiment is attached;

FIG. 15 is a photo illustrating a conventional fixing tool D for attaching an artificial ventilator (headgear for artificial ventilator (manufactured by RESMED));

FIG. 16 is a photo illustrating a fixing tool E for attaching an artificial ventilator of the present invention;

FIG. 17 is a schematic view illustrating an attaching spot of a thread when an attachment initial pressure is measured in an experiment in Example 2;

FIG. 18 is a schematic view illustrating a direction of a tensile force when the attachment initial pressure is measured in the experiment in Example 2;

FIG. 19 is a schematic view illustrating the direction of the tensile force and an attaching spot of a spring scale when a mask holding force is measured in the experiment in Example 2;

FIG. 20 is a graph showing a leak amount of a patient with sleep apnea syndrome in an experiment in Example 3, in which a lateral axis indicates a date and a vertical axis indicates the leak amount (L/min);

FIG. 21 is a graph showing AHI and AI of the patient with sleep apnea syndrome in an experiment in Example 4, in which a lateral axis indicates a date and a vertical axis indicates the AHI & AI (number of times/time); and

FIG. 22 is a graph showing time of use of the patient with sleep apnea syndrome in the experiment in Example 4, in which the lateral axis indicates a date and the vertical axis indicates time of use.

### DESCRIPTION OF EMBODIMENTS

Embodiments for performing the present invention will be described below in detail by using the attached drawings.

### First Embodiment of the Present Invention

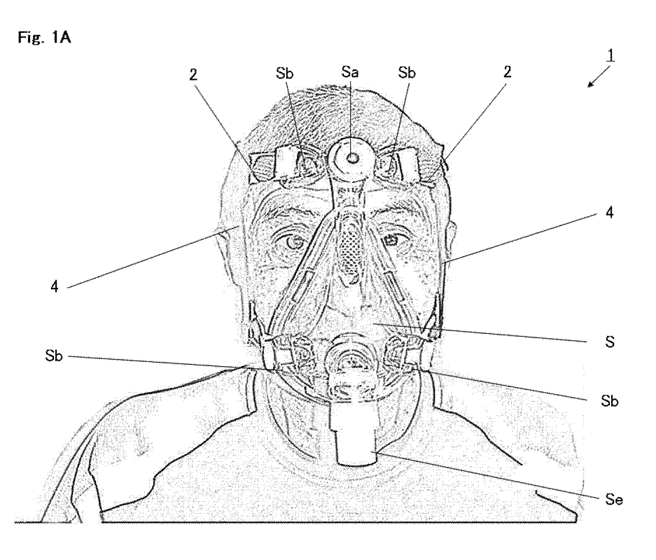

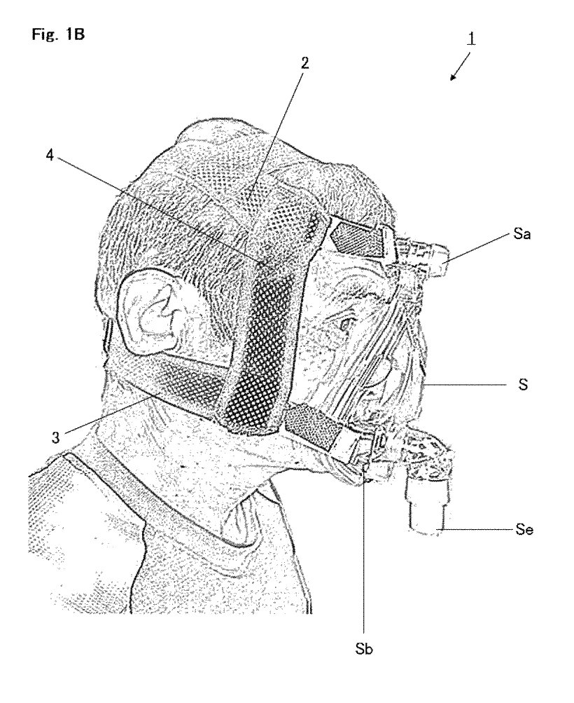

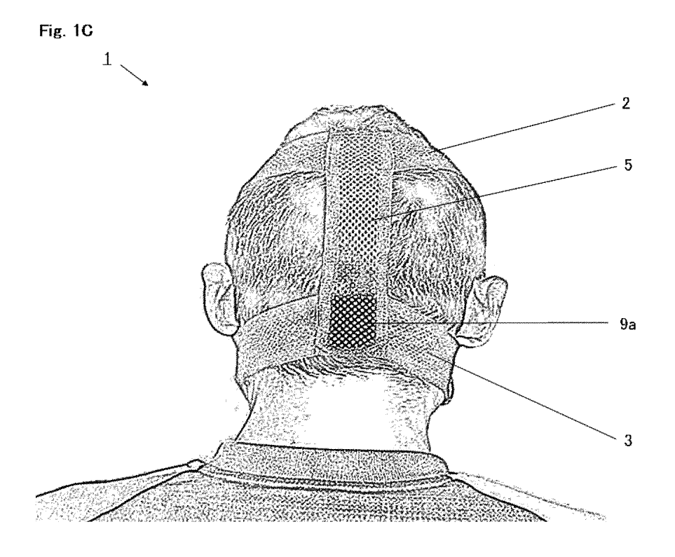

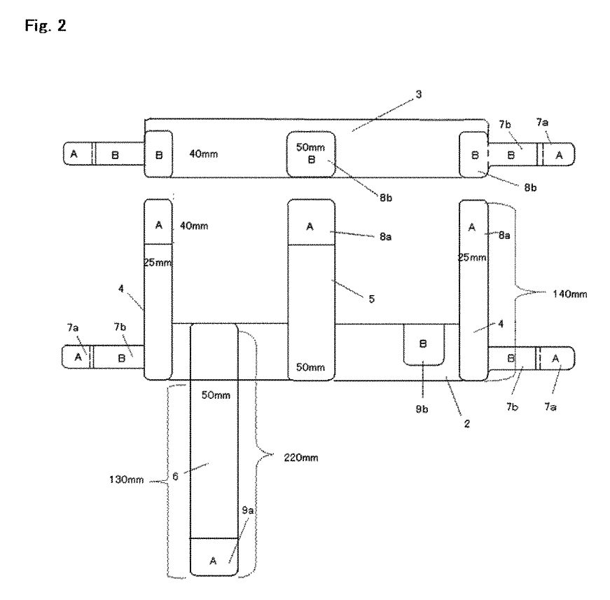

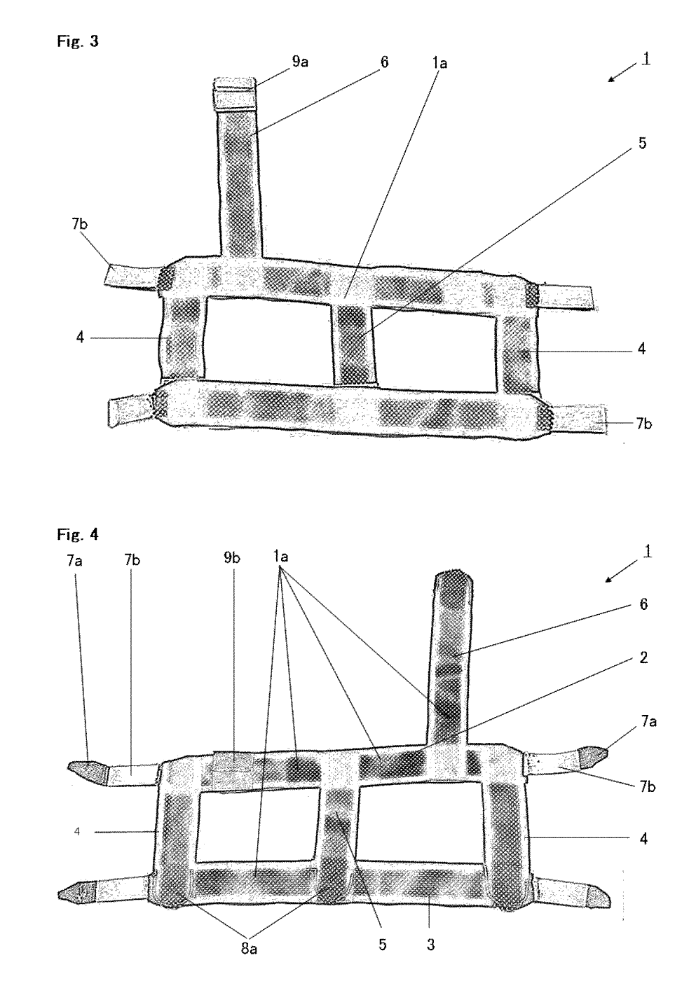

FIG. 1 is a perspective view exemplifying a fixing tool 1 for attaching an artificial ventilator of a first embodiment to which the present invention is applied. FIG. 2 is a development view of the fixing tool for attaching an artificial ventilator of the first embodiment.

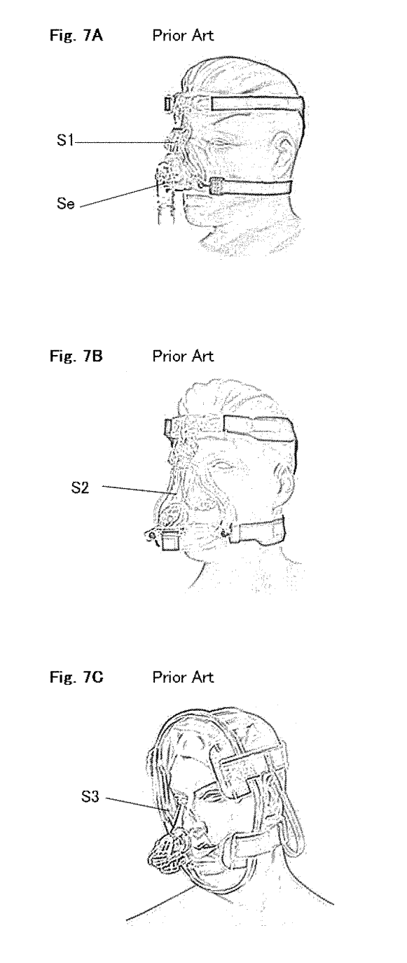

The fixing tool 1 for attaching an artificial ventilator of this embodiment comprises an upper-side band (forehead-side fixing band) for fixation in a circumferential direction from a forehead to a head and a lower side band (mouth-side fixing band) for fixation in the circumferential direction from a mouth to a neck part. These fixing bands 2 and 3 are mesh cloths with a large number of pores 1a formed, for which a woven fabric having a predetermined width and high stretchability is used, and the large number of pores 1a improve stretching performances and stretchability. For the fixing bands 2 and 3, a stretchable narrow-width woven fabric (narrow-width woven fabric with rubber), natural rubber, stretchable belt-like plastic, silicon rubber and the like can be used. Tip folding portions 7a and 7b of the fixing band are made into a folding length adjusting band. Attachment/detachment is made by using a hook-and-loop fastener. The length is adjusted by a holder Sb for connecting the folding length adjusting band and then, fixation is carried out. Right and left folding portions for fixing a center metal fitting are provided on right and left. Reference character Sa denotes a connection portion with a pressure/oxygen port, and reference character Se is a member called an elbow of a connection portion with the artificial ventilator. A nasal mask S1, a full-face mask S2, and a total face mask S3 are provided. Many of these fixing tools for medical artificial ventilation masks (headgear) include the forehead-side fixing band (upper-side band) for fixation in the circumferential direction from the forehead to the head and the mouth-side fixing band (lower-side band) for fixation in the circumferential direction from the mouth to the neck part (FIGS. 7A, 7B, and 7C). A single fixing band is used in some cases, but in order to ensure reliability of connection with the pressure/oxygen port and connection with the artificial ventilator, a double-band type, that is, upper and lower bands which are more stable and suppress occurrence of a displacement at an accurate position is on the mainstream.

The forehead-side fixing band 2 and the mouth-side fixing band 3 are disposed on the upper side (forehead-side fixing band) and the lower side (mouth-side fixing band) for fixation in the circumferential direction from the mouth to the neck part separately from each other (in a separated state) and are attached/detached by using predetermined connecting means (hook-and-loop fasteners) 8a, 8b 9a, and 9b via the connection band. By sewing these hook-and-loop fasteners 8a, 8b, 9a, and 9b at predetermined spots, connection by the connecting means only at the sewn spot is made possible. That is, the hook-and-loop fasteners 8a and 8b do not adhere to the portion of the large number of pores 1a in the mesh cloth. The large number of pores 1a in the mesh cloth are suitable as a member for preventing the hook-and-loop fasteners 8a and 8b from adhering.

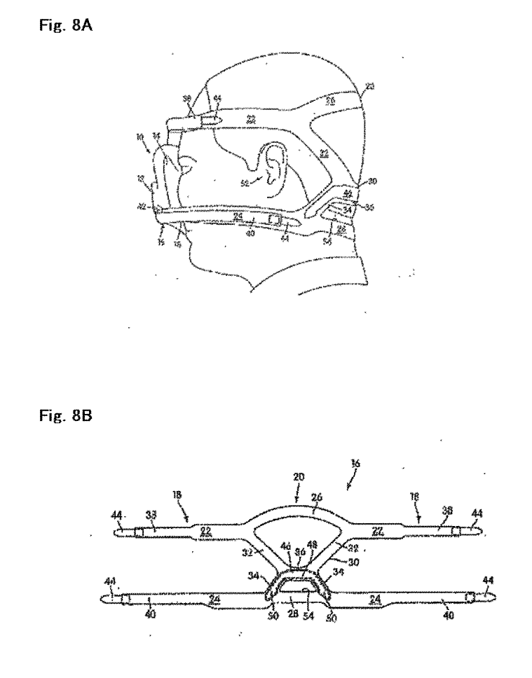

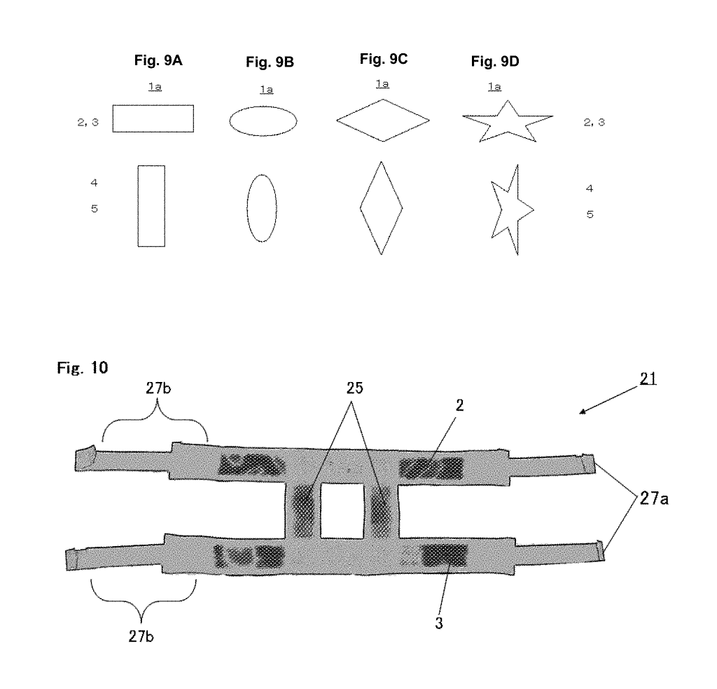

The forehead-side fixing band and the mouth-side fixing band 2 and 3 in a lateral direction and connection bands 4, 5, and 6 in a vertical direction are connected. That is, the head side connection band 6 connecting the forehead-side fixing band so as to cover the head, the side connection band 4 for connecting the forehead-side fixing band 2 and the mouth-side fixing band 3, and the head rear-side connection band 5 for holding the head rear side are connected. The jaw-part side connection band for connecting the mouth-side fixing band so as to cover a jaw part may be further provided (FIG. 8). For the head side connection band 6 and the right and left side connection bands 4 and the like, the same material as that of the forehead-side fixing band 2 and the mouth-side fixing band 3 is used. Therefore, air permeability is ensured by the mesh cloth, and more stretchability (stretchability in the vertical direction) is ensured by the large number of pores 1a. A shape of each of the large number of pores is preferably a rectangular shape, an elliptic shape, a diamond shape or a star-like shape along a longitudinal direction of each band of the stretchable cloth (stretchable fabric) (FIGS. 9A to 9D). That is because the stretchability in a longitudinal direction of each band is further exerted. And the rectangular shape, elliptic shape, diamond shape, star-like shape or the like is preferable (FIGS. 9A to 9D). When the forehead-side fixing band and the mouth-side fixing band 2 and 3 in the lateral direction and the connection bands 4, 5, and 6 in the vertical direction are connected, the large number of pores 1a each having a rectangular shape or the like formed along the longitudinal direction of these bands are disposed horizontally and vertically, whereby similar expanding force is exerted horizontally and vertically.

In this embodiment, each of the connection bands connected to the forehead-side fixing band 2 or the mouth-side fixing band 3 has a shape of a lattice (a state where gaps are formed in a square) or a shape in which one belt-like member (head side connection band 6) protrudes from the square. For example, the two head side connection bands 6 are disposed symmetrically so as to facilitate folding for storing/keeping, or front or rear of the head may be covered by the head side connection bands 6 so as to ensure stable fixation.

With the purpose of operating the artificial ventilator S1 as if a cap is worn, the number of the head side connection band 6 in this embodiment is one, but a plurality of them may be disposed or a wider one may be used.

The right and left side connection bands 4 are disposed so as to be located on side surfaces of the face closer to the center rather than to the ears in the crosswise direction. Therefore, by combining these fixing bands 4 and 4 with the forehead-side fixing band 2 and the mouth-side fixing band 3, they surround a square having a substantial size of the artificial ventilator S1 (only the front side of the face is surrounded by a small square), and the artificial ventilator S1 can be fixed to the front side of the face so as not to be displaced.

In this embodiment with the aforementioned constitution, the fixing tool can be mounted on a patient at a time like a cap, and can also be removed at a time.

The forehead-side fixing band 2 and the mouth-side fixing band 3 are constituted separably without having connecting means of their own and are attached/detached by using the hook-and-loop fasteners 8a, 8b, 9a, and 9b only by the predetermined connecting means via the connection bands. That is, in Patent Literature 1, the forehead-side fixing band 2 and the mouth-side fixing band 3 have crossing portions (see FIG. 9B), but in this embodiment, a crossing portion is not provided. Therefore, storing/keeping by folding into a square is easy, and folding is also easy.

Here, to a bed-ridden patient with an advanced disease, the connection band can be sequentially added and connected in accordance with the patient. That is, the forehead-side fixing band and the mouth-side fixing band are connected in advance to the artificial ventilator S, and the artificial ventilator S in this state is attached to the front of the face (from the mouth to the nose) of the patient. After the attachment, the connection band can be connected only at a position where the forehead-side fixing band and the mouth-side fixing band can be connected by the hook-and-loop fasteners 8a, 8b, 9a, and 9b. In this case, in a state where the side connection band 4 or the head rear-side connection band 5 is connected, some of them may be connected or the side connection band 4 and the head rear-side connection band 5 are not connected but the connection band may be connected by the hook-and-loop fasteners 8a, 8b, 9a, and 9b only at a predetermined spot. By combining the fixing bands made only of vertical and lateral lines into a lattice shape, in this embodiment, its stretching force is exerted also in a diagonal direction in addition to the vertical and lateral directions.

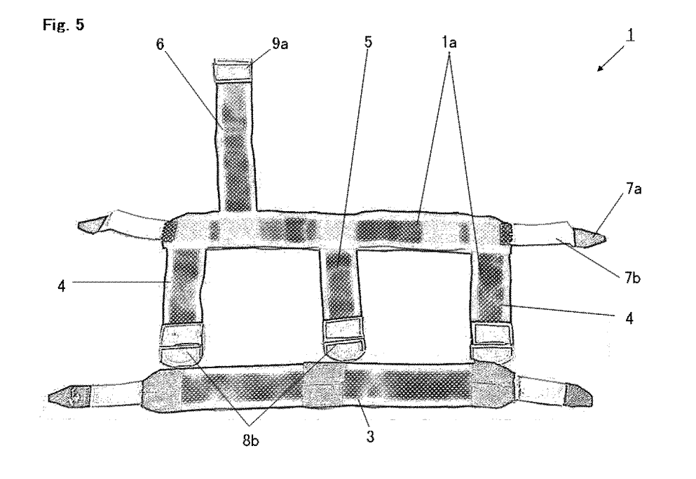

Moreover, by means of the connection bands 4, 5, and 6 for connecting the forehead-side fixing band 2 and the mouth-side fixing band 3, it is so constituted that attachment/detachment is made only via the connecting means such as the hook-and-loop fasteners 8a, 8b, 9a, and 9b. Thus, since only one side is connected by the connection bands 4, 5, and 6, the operation is easier than the connection on both sides of the connection bands 4, 5, and 6 (FIGS. 2 and 5). It is possible to connect the both sides of the connection bands 4, 5, and 6 depending on practice.

### Second Embodiment of the Present Invention

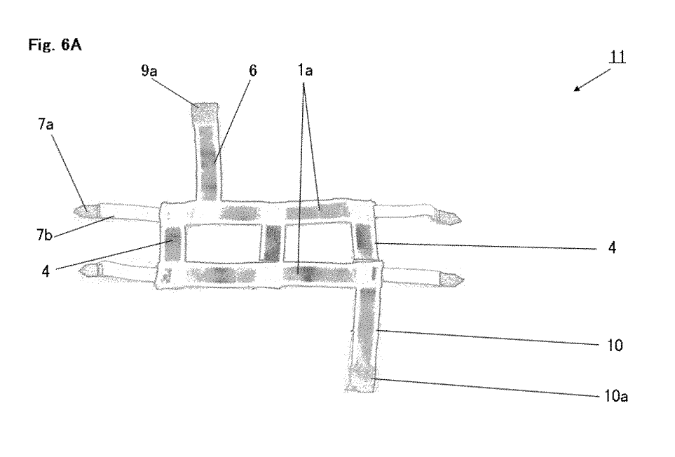

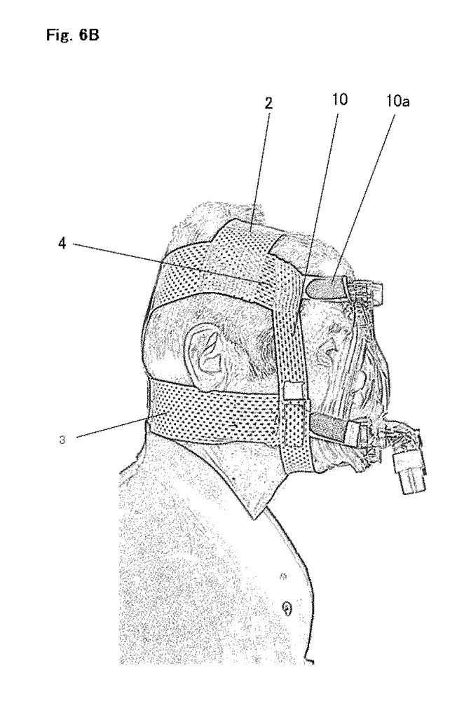

FIGS. 6A and 6B are views exemplifying a fixing tool 11 for attaching an artificial ventilator of a second embodiment to which the present invention is applied. In this embodiment, a jaw-part side connection band 10 for connecting the mouth-side fixing band 3 so as to cover the jaw part is further provided. Reference character 10a denotes a connection spot with the fixing band 3 by the hook-and-loop fastener. By adding the jaw-part side connection band 10, an entire shape looks like a semispherical cap for skiing, whereby an attaching/detaching operation is facilitated.

According to this embodiment, since there is a concern that the artificial ventilator (mask) S1 is displaced upward and removed depending on a patient (particularly during sleep), this situation of upward displacement can be prevented.

### Third Embodiment of the Present Invention

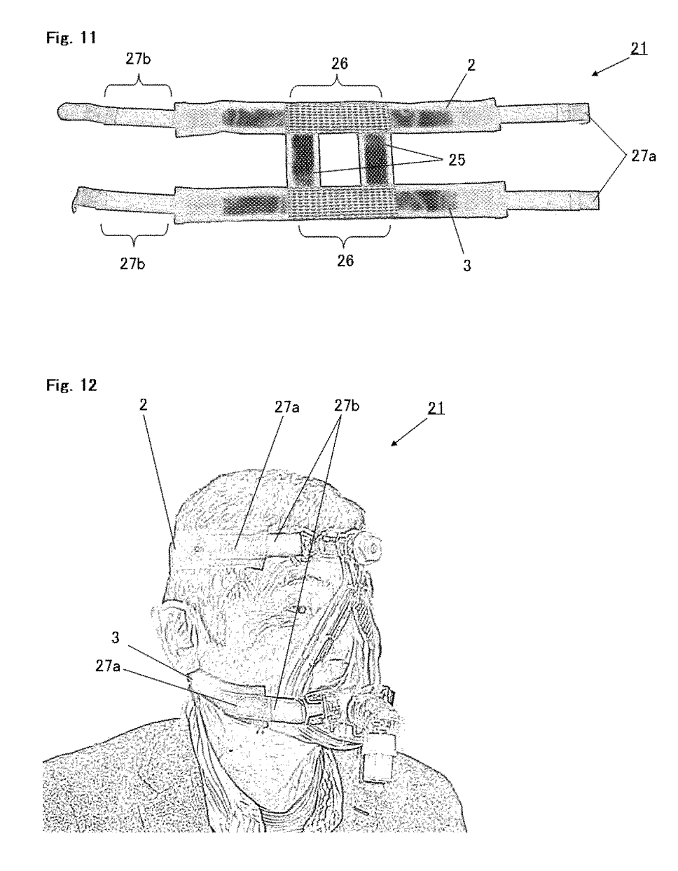

FIG. 10 is a rear view illustrating a fixing tool for attaching an artificial ventilator of a third embodiment to which the present invention is applied, and FIG. 11 is a front view illustrating the fixing tool for attaching an artificial ventilator of the third embodiment.

The fixing tool 1 for attaching an artificial ventilator of the first embodiment includes the side connection band 4, the head rear-side connection band 5, and the head side connection band 6, and the fixing tool 11 for attaching an artificial ventilator of the second embodiment further includes the jaw-part side connection band 10 in addition to the aforementioned connection bands 4, 5, and 6. A fixing tool 21 for attaching an artificial ventilator of the third embodiment has its constitution simplified and includes a pair of head rear-side connection bands 25 and a non-slip member 26 at front centers (spots where the head rear side is in contact) of the forehead-side fixing band 2 and the mouth-side fixing band 3. Since the other constitution is similar to those in the first and second embodiments, the same reference numerals are used for common structures, and duplicated description will be omitted.

The fixing tool 21 for attaching an artificial ventilator of this embodiment consists of the forehead-side fixing band 2, the mouth-side fixing band 3, the pair of head rear-side connection bands 25, and tip folding portions 27a and 27b.

The forehead-side fixing band 2 is for fixation by being wound in the circumferential direction from the forehead to an upper rear part of the head and fixes the artificial ventilator S in a state wound around the upper rear part of the head.

The mouth-side fixing band 3 is for fixation by being wound in the circumferential direction from the mouth to a lower rear part of the head and fixes the artificial ventilator S in a state wound around the lower rear part of the head.

The non-slip members 26 are fixed at the front center positions of the forehead-side fixing band 2 and the mouth-side fixing band 3, and the tip folding portions 27a and 27b are fixed at the right and left end portions on this figure.

The non-slip member 26 is for preventing such a situation that the forehead-side fixing band 2 and the mouth-side fixing band 3 slip from the head and the artificial ventilator S is displaced from the face front. It is formed of a non-slip material having a favorable wearing feeling for a user such as polyvinyl chloride, polyacrylic resin, polyurethane resin, silicon resin, polyester resin or the like, and a large number of pores 1a are formed so as to ensure air permeability. The large number of pores 1a communicate with the large number of pores (mesh cloth) 1a of the forehead-side fixing band 2 and the mouth-side fixing band 3 so as to ensure air permeability.

The non-slip member 26 provides a favorable wearing feeling when it touches the rear occipital part due to presence of hair, but if the non-slip member 26 touches the skin on the side surface of the head of the user, it is likely that friction occurs between the skin and the non-slip member 26 and damages the skin. Thus, fixed positions of the non-slip members 26 are fixed to the front center positions of the forehead-side fixing band 2 and the mouth-side fixing band 3 not in contact with the skin.

The tip folding portions 27a and 27b are fixed to fixing-tool attaching members provided on upper, lower, right and left ends of the artificial ventilator S and are constituted by detachable members such as hook-and-loop fasteners. The tip folding portion 27a is a hook-shaped member, and the tip folding portion 27b is a loop-shaped member. The tip folding portion 27b and the tip folding portion 27a are fixed on the ends of forehead-side fixing band 2 and the mouth-side fixing band 3 in this order from the right to left on this figure, and they are adhered to each other by inserting the tip folding portion 27a through the fixing-tool attaching member of the artificial ventilator S, folding it back to a rear surface side, and pressing the folded tip folding portion 27a onto the tip folding portion 27b. By adjusting the folding position of the tip folding portion 27a, a length when the fixing tool 21 for attaching an artificial ventilator was attached to the artificial ventilator S can be changed, and adjustment according to the size of the head of the user is made possible.

The tip folding portion 27b is formed lengthily, assuming adjustment according to the size of the head of the user as described above and is fixed in a state where the both end portions of the forehead-side fixing band 2, the mouth-side fixing band 3 and the tip folding portions 27b are overlapped.

The pair of head rear-side connection bands 25 is fixed in the vicinity of the centers of the forehead-side fixing band 2 and the mouth-side fixing band 3 substantially perpendicularly at an interval and disposed so as to span from the forehead-side fixing band 2 to the mouth-side fixing band 3. The pair of head rear-side connection bands 25 spanning as above fixes the forehead-side fixing band 2 and the mouth-side fixing band 3 so as not to be displaced on the head rear side and also fixes the artificial ventilator S together with the forehead-side fixing band 2 and the mouth-side fixing band 3 to the face front when the artificial ventilator S is attached.

The forehead-side fixing band 2, the mouth-side fixing band 3, and the pair of head rear-side connection bands 25 are mash cloths on which the large number of pores 1a are formed and whose stretching performances and stretchability are improved by the large number of pores 1a, and a stretchable narrow-width woven fabric (narrow-width woven fabric comprising rubber), natural rubber, stretchable belt-like plastic, silicon rubber or the like can be used. Air permeability is ensured by the mesh cloth, and the stretchability (stretchability in the vertical and horizontal directions) is further ensured by the large number of pores 1a. They continue to the large number of pores 1a in the non-slip member 26, and the air permeability is ensured.

A joining method between the tip folding portion 27a and the tip folding portion 27b, a joining method among the tip folding portion 27b, the forehead-side fixing band 2, and the mouth-side fixing band 3, a joining method among the pair of head rear-side connection bands 25, the forehead-side fixing band 2, and the mouth-side fixing band 3, and a joining method among the non-slip member 26, the forehead-side fixing band 2, and the mouth-side fixing band 3 can be selected as appropriate from thermocompression bonding, sewing, laminating or the like.

Moreover, the pair of head rear-side connection bands 25, the forehead-side fixing band 2 and the mouth-side fixing band 3 may be detachably fixed by attaching/detaching means such as a hook-and-loop fastener. If they are detachably fixed, the artificial ventilator S can be attached/detached more easily by removing the pair of head rear-side connection bands 25 from the forehead-side fixing band 2 and the mouth-side fixing band 3.

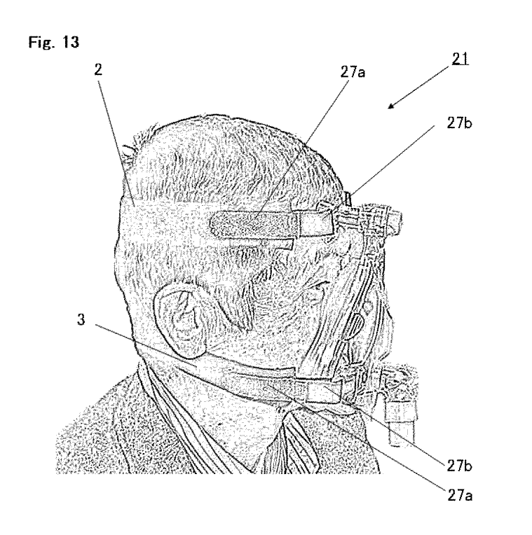

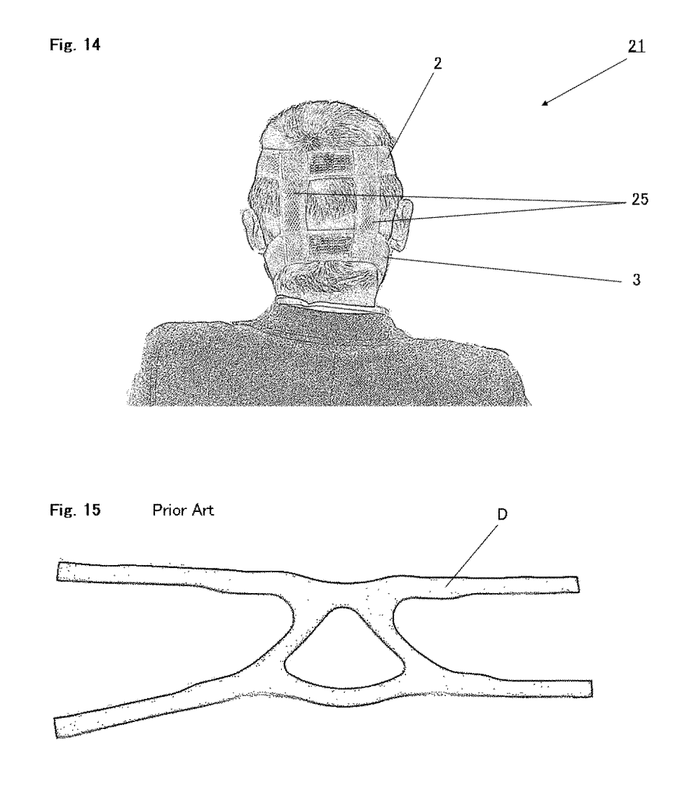

FIG. 12 is a perspective view of a state where the fixing tool for attaching an artificial ventilator of the third embodiment is attached, and FIG. 13 is a side view of a state where the fixing tool for attaching an artificial ventilator of the third embodiment is attached. FIG. 14 is a rear view of a state where the fixing tool for attaching an artificial ventilator of the third embodiment is attached.

In this embodiment with the aforementioned constitution, the fixing tool can be mounted on a patient at a time like a cap, and can also be removed at a time.

Moreover, air permeability is ensured since the pair of head rear-side connection bands 25, the forehead-side fixing band 2, and the mouth-side fixing band 3 are formed of a mesh cloth on which a large number of pores 1a are formed, and stretchability (stretchability in vertical and horizontal directions) is improved by the large number of pores 1a so that a wearing feeling is favorable and attachment without displacement of the artificial ventilator S is realized even if a user moves.

In addition, since the non-slip member 26 is fixed at the front center positions of the forehead-side fixing band 2 and the mouth-side fixing band 3, the artificial ventilator S can be favorably attached for a long time without displacement.

Moreover, since the number of members constituting the fixing tool 21 for attaching an artificial ventilator is small, inexpensive manufacture is realized.

The non-slip member may be provided in the fixing tools 1 and 11 for attaching an artificial ventilator in the first and second embodiments. When the non-slip member is used in the fixing tools 1 and 11 for attaching an artificial ventilator in the first and second embodiments, it is important that the non-slip member should be attached at a spot where the non-slip member does not touch the skin such as the front of the head rear-side connection band 5 or the front center positions of the forehead-side fixing band 2 and the mouth-side fixing band 3 and the like.

### Example 1

In Example 1, fixing tools A, B, and C for attaching an artificial ventilator were made, and experiments of a wearing feeling and the like were conducted.

In the fixing tool A, each of the fixing bands 2 and 3 has a length (excluding the tip folding portions 7a, 7b, 27a, and 27b) of 320 mm and a width of 50 to 65 mm, and each of the right and left side connection bands 4 has a length (including the connecting portions 8a and 8b) of 140 mm and a width of 25 mm A head side connection band 6 has a length (including the connecting portions 8a and 8b) of 130 mm and a width of 25 mm And they are constituted by a belt-like member made of a stretchable cloth having the stretch rate of 140 to 180%. Here, the stretch rate means an elongation rate at a 0.5 kg load (constant load method) described in JIS L1096.

The fixing tool B is slightly smaller than the fixing tool A, and each of the fixing bands 2 and 3 has a length (excluding the tip folding portions 7a, 7b, 27a, and 27b) of 300 mm and a width of 30 to 45 mm, and each of the right and left side connection bands 4 has a length (including the connecting portions 8a and 8b) of 120 mm and a width of 20 mm A head side connection band 6 has a length (including the connecting portions 8a and 8b) of 110 mm and a width of 25 mm And they are constituted by a belt-like member made of a stretchable fabric having the stretch rate of 200 to 300%.

The fixing tool C was manufactured by using a stretchable belt-like plastic material which is not a mesh cloth on which the large number of pores 1a are formed, although the length and the width are the same as those of the fixing tool B. The stretch rate was 100 to 200%. Those having the stretch rate less than 100% and larger than 300% were made, respectively, and used as comparative examples. In this embodiment, the nasal mask was used for the artificial ventilator S1 and moreover, it is used also for full-face mask S2 and the total-face mask S3 in the experiments.

First, if the stretch rate is less than 100%, the feeling at attachment/detachment of the artificial ventilator S1 is slightly loose, while if the stretch rate is larger than 300%, the tightening feeling is strong. In this embodiment, the length of each of the connection bands 4, 5, and 6 can be adjusted by the hook-and-loop fastener even in such cases.

In all the fixing tools A, B, and C, stretchability is improved by the large number of pores 1a in the mesh cloth and thus, they can be applied even with a stretch rate weaker than the stretch rate of the cloth which is not a mesh cloth. That is, even if the stretch rate is weaker by 10 to 30% than the aforementioned stretch rates of 100 to 300%, there is no problem with the wearing feeling, and even if the stretch rate is weaker by 10 to 30% than the aforementioned stretch rates of 100 to 300%, the wearing feeling and the attachment/detachment operation were favorable.

In the fixing tool A and the fixing tool B made of the mesh cloth, it was known that stretchability was exerted not only in the vertical direction and in the lateral direction but also in directions such as a diagonal direction as compared with the fixing tool C not made of the mesh cloth, and the entirety exerts flexibility, and the wearing feeling and the attachment/detachment operation were favorable. The large number of pores 1a in each of the fixing tool A and the fixing tool B allow the cloth to exert the stretching force, but it was known that, by connecting and assembling the forehead-side fixing band and the mouth-side fixing band 2 and 3 in the lateral direction and the connection bands 4, 5, and 6 in the vertical direction, the stretching force was exerted also in the diagonal direction through the large number of pores 1a. It is considered that the stretching force is exerted entirely as a cap made of wool (a cap for winter), for example.

From the aforementioned Example 1, the fixing tool is preferably constituted by the belt-like member made of a stretchable cloth having the stretch rate of 100 to 300%, and a stretchable mesh cloth having the stretch rate of 140 to 180% with a large number of pores formed is particularly preferable. It should be noted that the stretch rate means an elongation rate at a 0.5 kg load (constant load method) described in JIS L1096. A calculation formula of the stretch rate is 100×B/A, where B is a stretched entire length and A is an original length (in the calculation formula, the stretch rate is 100% when actual elongation is 0). By combining each of the connection bands 4, 5, and 6 with the forehead-side fixing band 2 and the mouth-side fixing band 3, the fixing tool according to the present invention can be assembled so that stretchability is exerted not only in vertical and horizontal directions but also in a diagonal direction, and that the artificial ventilator is disposed on the front of the face by wearing it at a time like a cap and is also removed at a time. By providing the large number of pores 1a in the mesh cloth of the fixing tool 1 for attaching an artificial ventilator as in this embodiment, the washing effect also becomes higher.

The aforementioned fixing tools A, B, and C can be applied to any one of the nasal mask (artificial ventilator) S1, the full-face mask S2, and the total-face mask S3, but the fixing tool A having a relatively wide width is more preferably applied to the full-face mask S2 and the total-face mask S3. In the case of a patient in home care or a patient for whom a little more pressure is preferably applied, although the patient has got out of an acute phase, since connection by the hook-and-loop fasteners 8a, 8b, 9a, and 9b limit the contact only at that spot (since the hook-and-loop fastener does not join easily to the spot with the large number of pores), a work of attachment/detachment of the artificial ventilator S1 can be carried out easily.

### Example 2

In Example 2, a comparative experiment of a holding force was conducted by using the conventional fixing tool for attaching an artificial ventilator and the fixing tool for attaching an artificial ventilator of the present invention.

(Employed Members)

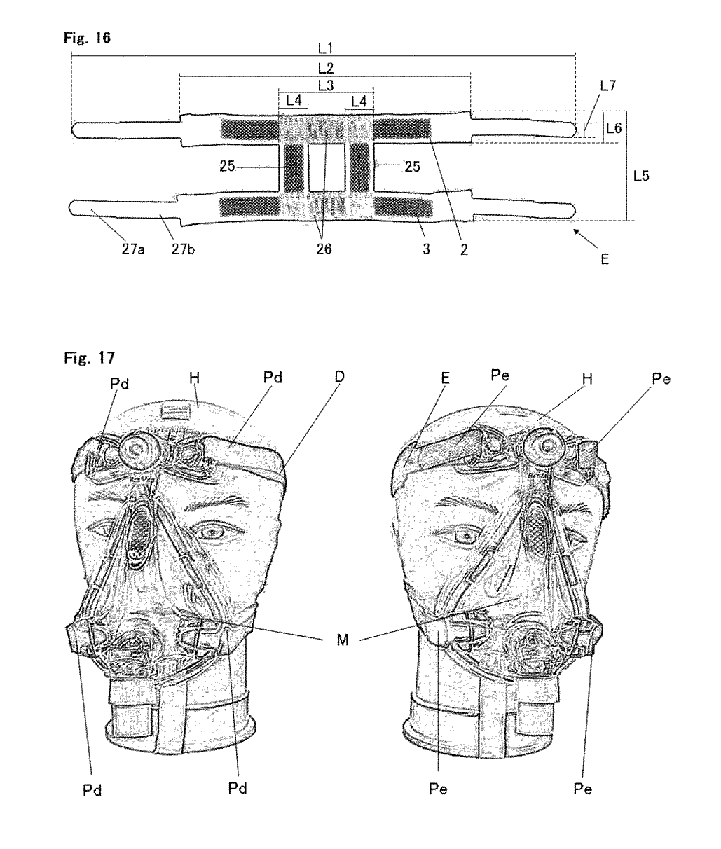

FIG. 15 is a photo illustrating a conventional fixing tool D for attaching an artificial ventilator (headgear for artificial ventilator (manufactured by RESMED)), and FIG. 16 is a photo illustrating a fixing tool E for attaching an artificial ventilator of the present invention.

As the conventional fixing tool for attaching an artificial ventilator, a headgear for artificial ventilator (manufactured by RESMED)) (fixing tool D for attaching an artificial ventilator) (FIG. 15) was used.

As the fixing tool for attaching an artificial ventilator of the present invention, the fixing tool E for attaching an artificial ventilator (FIG. 16) described in the third embodiment of the present invention was used. An entire length L1 of the fixing tool E for attaching an artificial ventilator is approximately 63.0 cm, a width L5 of the fixing tool E for attaching an artificial ventilator is approximately 13.5 cm, a length L2 of each of the forehead-side fixing band 2 and the mouth-side fixing band 3 is approximately 35.0 cm, a width L6 of each of the forehead-side fixing band 2 and the mouth-side fixing band 3 is approximately 4.0 cm, a length L3 of the non-slip member 26 is approximately 11.0 cm, a width L4 of the head rear-side connection band 25 is approximately 3.5 cm, and a width L7 of each of the tip folding portions 27a and 27b is approximately 2.0 cm.

As masks M to which the fixing tools D and E for attaching an artificial ventilator are attached, the full-face mask (manufactured by RESMED) and the nasal mask (manufactured by RESMED) were used.

As a pseudo head H to which the mask on which the fixing tool D or E for attaching an artificial ventilator is installed is to be attached, a model head made of a material of styrene foam with a head circumference of 55 cm and a length of the face of 20 cm was used.

(Experiment Method)

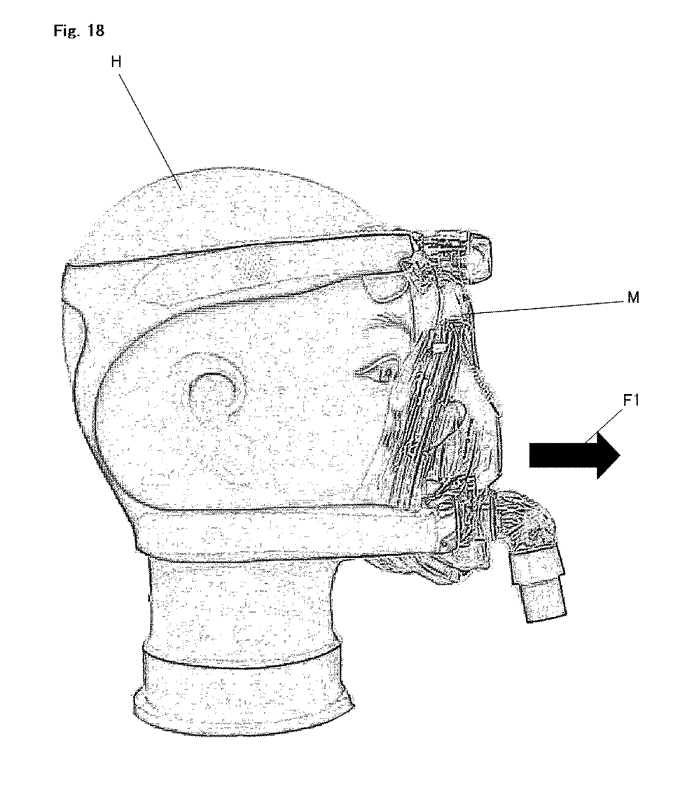

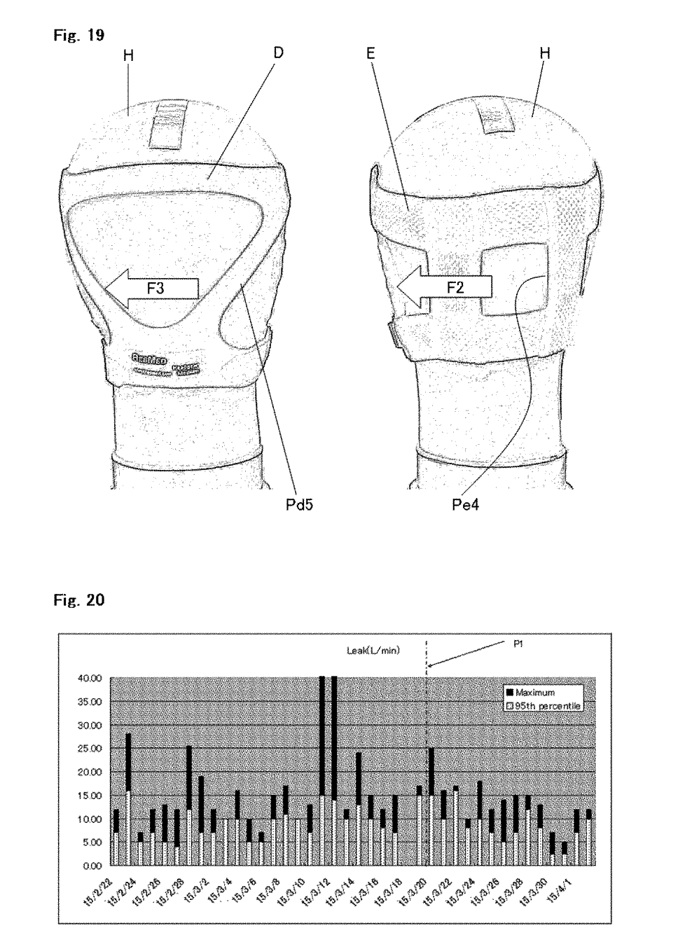

FIG. 17 is a schematic view illustrating an attachment spot of a thread when an attachment initial pressure is measured in the experiment of Example 2, and FIG. 18 is a schematic view illustrating a direction of a tensile force when the attachment initial pressure is measured in the experiment of Example 2. FIG. 19 is a schematic view illustrating the direction of the tensile force and an attaching spot of a spring scale when a mask holding force is measured in the experiment in Example 2.

In the experiment of the holding force, the following measurements (1) to (3) were made.

The fixing tool D or E for attaching an artificial ventilator was installed on the mask M, and in a state where the mask M and the fixing tools D or E for attaching an artificial ventilator are attached to the pseudo head H, the attachment initial pressure, the mask holding force, and a maximum elongation were measured by a spring scale. Moreover, as the type of the mask M, measurement was made for each of the full-face mask and the nasal mask.

(1) Attachment initial pressure: One ends of threads were attached to each of four spots (Pd1, Pd2, Pd3, Pd4) of attaching spots between the mask M and the fixing tool D for attaching an artificial ventilator, and the other ends of the threads were gathered to a spot in the periphery of a front center of the mask M and bound. Similarly, one ends of threads were attached to each of four spots (Pe1, Pe2, Pe3, Pe4) of attaching spots between the mask M and the fixing tool E for attaching an artificial ventilator, and the other ends of the threads were gathered to a spot in the periphery of a front center of the mask M and bound (FIG. 17). The spring scale was applied at the spot where the threads were bound and pulled in an F1 direction (FIG. 18), and the tensile force at a moment when the mask M left the face was measured by the spring scale for each of the fixing tools D and E for attaching an artificial ventilator.

(2) Mask holding force: The spring scale was applied at two spots, that is, Pd5 an Pe5 of the fixing tools D and E for attaching an artificial ventilator, respectively, and pulled in F2 and F3 directions and an average tensile force (kg), a minimum tensile force (kg), and a maximum tensile force (kg) at a moment when the mask M was displaced were measured by the spring scale, respectively (FIG. 19).

(3) Maximum elongation: One ends of threads were attached to each of the four spots (Pd1, Pd2, Pd3, Pd4) of the attaching spots between the mask M and the fixing tool D for attaching an artificial ventilator, while the other ends of the threads were gathered to a spot in the periphery of a front center of the mask M and bound. Similarly, one ends of threads were attached to each of four spots (Pe1, Pe2, Pe3, Pe4) of attaching spots between the mask M and the fixing tool E for attaching an artificial ventilator, and the other ends of the threads were gathered to a spot in the periphery of a front center of the mask M and bound (FIG. 17). When the spring scale was applied at the spot where the threads were bound and pulled in the F1 direction (FIG. 18) with a force of 4.5 kg, the mask M left the face. If the mask M leaves the face, an interval (elongation length) between the center of the face and the center of the mask M is measured.

(Experiment Result)

Experiment results of the holding forces in the aforementioned (1) to (3) are shown in Table 1 and Table 2 below. Table 1 is the experiment result of the holding force when the nasal mask was used, and Table 2 is the experiment result of the holding force when the full-face mask was used.

TABLE 1

| | | mask holding force | | | | |
|---|---|---|---|---|---|---|
| | | average | minimum | maximum | | |
| | initial | tensile force | tensile force | tensile force | | |
| | attachment | at a moment | at a moment | at a moment | maximum elongation | |
| | pressure | when the mask | when the mask | when the mask | maximum | elongation |
| | tensile force | is displaced | is displaced | is displaced | pressure | length |
| | (kg) | (kg) | (kg) | (kg) | (kg) | (mm) |
| conventional fixing | 2.0 | 2.5 | 2.0 | 3.0 | 4.5 | 20.0 |
| tool D for attaching | | | | | | |
| artificial ventilator | | | | | | |
| fixing tool E | 2.0 | 3.0 | 2.5 | 3.5 | 4.5 | 50.0 |
| for attaching artificial | | | | | | |
| ventilator | | | | | | |

TABLE 2

| | | mask holding force | | | | |
|---|---|---|---|---|---|---|
| | | average | minimum | maximum | | |
| | initial | tensile force | tensile force | tensile force | | |
| | attachment | at a moment | at a moment | at a moment | maximum elongation | |
| | pressure | when the mask | when the mask | when the mask | maximum | elongation |
| | tensile force | is displaced | is displaced | is displaced | pressure | length |
| | (kg) | (kg) | (kg) | (kg) | (kg) | (mm) |
| conventional fixing | 2.0 | 4.0 | 3.5 | 4.5 | 4.5 | 10.0 |
| tool D for attaching | | | | | | |
| artificial ventilator | | | | | | |
| fixing tool E | 2.0 | 4.5 | 4.0 | 4.8 | 4.5 | 40.0 |
| for attaching artificial | | | | | | |
| ventilator | | | | | | |

As shown in Table 1 and Table 2, when the “mask holding force” is focused, since the fixing tool E for attaching an artificial ventilator of the present invention has an average tensile force larger than that of the conventional fixing tool D for attaching an artificial ventilator, it turned out that the mask is not displaced easily in the fixing tool E for attaching an artificial ventilator of the present invention against the force from an outside. The reason why the mask is not displaced easily in the fixing tool E for attaching an artificial ventilator of the present invention as described above (the mask holding force is improved) is that, as shown in the “maximum elongation” in Table 1 and Table 2, the fixing tool E for attaching an artificial ventilator of the present invention has the “elongation length” of 2.5 to 4.0 times longer than that of the conventional fixing tool D for attaching an artificial ventilator and its stretchability is extremely excellent.

### Example 3

In Example 3, a patient with sleep apnea syndrome actually wore the conventional fixing tool for attaching an artificial ventilator and the fixing tool for attaching an artificial ventilator of the present invention and used them for several days, and the wearing feeling and the leak amount were checked.

(Employed Members)

As the conventional fixing tool for attaching an artificial ventilator and the fixing tool for attaching an artificial ventilator of the present invention, the members similar to those in Example 2 were employed.

(Experiment Method)

The conventional fixing tool for attaching an artificial ventilator (use period: Feb. 22, 2015 to Mar. 19, 2015) and the fixing tool for attaching an artificial ventilator of the present invention (use period: Mar. 20, 2015 to Apr. 3, 2015) were applied to the patient with sleep apnea syndrome during treatment, and the patient was interviewed about a difference in the wearing feeling, and the leak amounts were measured.

(Experiment Result)

FIG. 20 is a graph showing the leak amount of the patient with sleep apnea syndrome in the experiment in Example 3, in which a lateral axis indicates a date and a vertical axis indicates the leak amount (L/min).

Treatment was carried out, in which the conventional fixing tool for attaching an artificial ventilator was replaced with the fixing tool for attaching an artificial ventilator of the present invention at a point of time P1 in FIG. 20, and it is known that the leak amount during the period when the fixing tool for attaching an artificial ventilator of the present invention is used (use period: Mar. 20, 2015 to Apr. 3, 2015) is decreased as compared with the leak amount during a period when the conventional fixing tool for attaching an artificial ventilator is used (use period: Feb. 22, 2015 to Mar. 19, 2015).

As the result of the interview on the wearing feeling, the fixing tool for attaching an artificial ventilator of the present invention seemed that the fixing tool was more elastic and an attachment pad installed on the mask fitted the face better and gave a feeling of close contact as compared with the conventional fixing tool for attaching an artificial ventilator. While the conventional fixing tool for attaching an artificial ventilator left attachment traces of the fixing tool on the face when the patient woke up in the morning in many cases, the fixing tool for attaching an artificial ventilator of the present invention left little trace and the patient had an impression that the wearing feeling was good and the like.

### Example 4

In Example 4, the conventional fixing tool for attaching an artificial ventilator and the fixing tool for attaching an artificial ventilator of the present invention were actually attached to a patient with sleep apnea syndrome and used for several days, and changes in AHI (apnea hypopnea index) and AI (apnea index) were measured.

Here, AHI (apnea hypopnea index) indicates the number of times combining apnea and hypopnea per hour of sleep, and AI (apnea index) indicates the number of times of apnea per hour of sleep.

(Employed Members)

As the conventional fixing tool for attaching an artificial ventilator and the fixing tool for attaching an artificial ventilator of the present invention, the members similar to those in Example 2 were employed.

(Experiment Method)

The conventional fixing tool for attaching an artificial ventilator (use period: Mar. 13, 2015 to Apr. 8, 2015) and the fixing tool for attaching an artificial ventilator of the present invention (use period: Apr. 9, 2015 to Apr. 21, 2015) were applied to the patient with sleep apnea syndrome during treatment, and the changes in AHI (apnea hypopnea index) and AI (apnea index) were measured.

(Experiment Result)

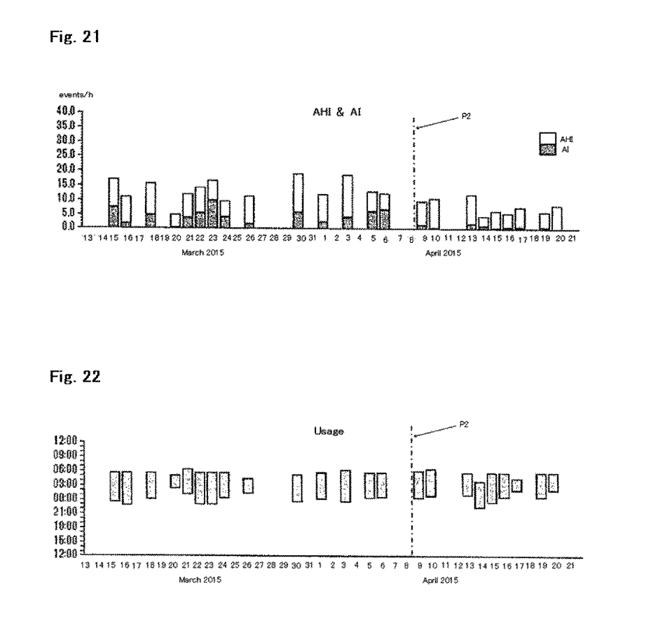

FIG. 21 is a graph showing the AHI and AI of the patient with sleep apnea syndrome in the experiment in Example 4, in which the lateral axis indicates a date and the vertical axis indicates the AHI & AI (number of times/hour), and FIG. 22 is a graph showing time of use of the patient with sleep apnea syndrome in the experiment in Example 4, in which the lateral axis indicates a date and the vertical axis indicates the time of use.

Treatment was carried out, in which the conventional fixing tool for attaching an artificial ventilator was replaced with the fixing tool for attaching an artificial ventilator of the present invention at a point of time P2 (Apr. 9, 2015) in FIGS. 21 and 22. It is known from the graph in FIG. 21, values of the AHI and AI during the period when the fixing tool for attaching an artificial ventilator of the present invention was used are smaller than the AHI and AI during the period when the conventional fixing tool for attaching an artificial ventilator was used. From the aforementioned results, it can be considered that using the fixing tool for attaching an artificial ventilator of the present invention results in appropriate fit of the mask onto the face due to its extremely excellent stretchability than the conventional fixing tools for attaching an artificial ventilator, so that the mask could be hardly displaced and air leakage (leak amount) from the mask was decreased, and thus the apnea hypopnea index or apnea index (AHI, AI) was decreased.

The case where the nasal mask is used as an artificial ventilator is described in this embodiment, but the present invention can be also applied to the full-face mask S2, the total-face mask S3 and the like. Although the large number of pores 1a in the mesh cloth each having a rectangular shape or an elliptic shape and the like are described, the shape is not limited. Thus, a regular square or other shapes may be employed.

### REFERENCE NUMERALS

1, 11, 21 fixing tool for attaching artificial ventilator
1a a large number of pores on mesh cloth
2 forehead-side fixing band (upper side fixing band)
3 mouth-side fixing band (lower side fixing band)
4 side connection band (connection band)
5, 25 head rear-side connection band (connection band)
6 head side connection band (connection band)
8a, 8b, 9a, 9b connecting means (hook-and-loop fastener)
10 jaw-part side connection band (connection band)
26 non-slip member
S, S1, S2, S3 artificial ventilator

1. A fixing tool for attaching an artificial ventilator for attaching/detaching the artificial ventilator to/from a front side of a face by using a fixing band formed of a stretchable narrow-width woven fabric, wherein
the fixing band comprises:
a forehead-side fixing band for fixation in a circumferential direction from a forehead to a head;
a mouth-side fixing band for fixation in the circumferential direction from a mouth to a neck part;
a head rear-side connection band and/or a side connection band for fixing the forehead-side fixing band and the mouth-side fixing band so as not to be displaced on the head; and
a slip resistant member fixed to center positions of the forehead-side fixing band and the mouth-side fixing band and on which air-permeable pores are formed.

2-8. (canceled)

9. The fixing tool for attaching an artificial ventilator according to claim 1, wherein
the head rear-side connection band is fixed substantially perpendicularly at the vicinity of the center of the mouth-side fixing band and disposed so as to span from the forehead-side fixing band to the mouth-side fixing band.

10. The fixing tool for attaching an artificial ventilator according to claim 1, wherein
the fixing band is constituted by a belt-like member made of a stretchable cloth which is a mesh cloth with a large number of pores formed and which has the stretch rate of 140 to 180%.

11. The fixing tool for attaching an artificial ventilator according to claim 1, wherein
the fixing band further comprises a jaw-side connection band for connecting the mouth-side fixing band so as to cover a jaw part.

12. The fixing tool for attaching an artificial ventilator according to claim 1, wherein
the fixing band further comprises a head side connection band for connecting the forehead-side fixing band so as to cover the head.

13. The fixing tool for attaching an artificial ventilator according to claim 1, wherein
the fixing band is provided with a large number of pores, and
a shape of the pore is a shape along a longitudinal direction of the fixing band.

14. The fixing tool for attaching an artificial ventilator according to claim 13, wherein
the shape of the pore is a elliptic shape or a star-like shape along the longitudinal direction of the fixing band.

15. The fixing tool for attaching an artificial ventilator according to claim 13, wherein
the shape of the pore is a rectangular shape or a diamond shape along the longitudinal direction of the fixing band.

16. The fixing tool for attaching an artificial ventilator according to claim 1, wherein
the stretchable narrow-width woven fabric is a narrow-width woven fabric comprising rubber.

17. The fixing tool for attaching an artificial ventilator according to claim 1, wherein
the slip resistant member is formed of polyvinyl chloride.

18. The fixing tool for attaching an artificial ventilator according to claim 1, wherein
the forehead-side fixing band and the mouth-side fixing band are attached/detached by the connection band for connecting them only on one sides thereof via the connecting tool such as a hook-and-loop fastener, and the other sides are connected in advance by sewing or the like.

19. The fixing tool for attaching an artificial ventilator according to claim 1, wherein
the fixing tool for attaching the artificial ventilator is used for treatment of a patient with a sleep apnea syndrome.
